(19) United States
(12) Patent Application Publication
(10) Pub. No.: US 2004/0237143 A1
(43) Pub. Date: Nov. 25, 2004

(54) Seed composition and method for reducing and preventing the release of genetically manipulated pollen

(76) Inventors: Peter Stamp, Zurich (CH); Boy Feil, Dubendorf (CH)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/333,165

(22) PCT Filed: Jul. 16, 2001
(86) PCT No.: PCT/CH01/00440

(30) Foreign Application Priority Data
Jul. 17, 2000 (EP) 00810631.2

(51) Int. Cl. A01H 1/00; C12N 15/82

(52) U.S. Cl. 800/287

(57) ABSTRACT

The present invention relates to a seed composition and a method for reducing and, if desired, for preventing the release of pollen or functional pollen from genetically modified crops by cultivating fields stands of plants bearing target GMO traits in a male sterile version, i.e. without the ability to produce pollen or functional pollen.

The present invention relates to a seed composition and a method for reducing and for preventing the release of pollen from genetically modified (GMO), i.e. transgenic plants by cultivating GMO plants on farmer's fields in a male sterile version, i.e. without the ability to produce pollen or functional pollen.

Plants, which are grown for grain production usually release a high number of pollen. Cross-pollinating crops such as maize (Zea mays L.) call produce several million pollen grains. Under favourable conditions, the pollen grains remain viable for up to 24 hours and can be transported on the airflow over longer distances. Pollen dispersal away from the vicinity of the crops can also take place by carriage on insects such as bees. In this way, pollen can be transported several kilometres from the crop plot.

Pollen dispersal has long been of interest in seed production as the potential exists for contamination of one crop version with the pollen of another. Interest in pollen dispersal has recently been renewed with the advent of GMO plants for several reasons:

Pollen from fields planted with GMO plants may fertilize plants in fields planted with wildtype plants, which results in grains containing the transgene even though no effects are visible. Such products may create problems when thresholds are set for the content of transgenes in seeds and grains. For example, transgenes are not tolerated in organically grown goods

Negative impacts of GMO pollen on non-target organisms. For example, it has been reported that monarch butterfly larvae fed milkweed leaves artificially coated with pollen from Bt maize ate less, grew slower, and suffered a higher death rate than larvae that consumed milkweed leaves free of maize pollen (Losey et al. 1993).

GMO pollen in honey There is evidence that even maize pollen is collected by honey bees in notable amounts (Hodges 1984)

Transfer of transgenes to bacteria in the intestines of bees (H. H. Kaatz, University Jena, and S. Wölfl, Hans Knoll Institut far Naturstoff-Forschung, Jena: RP-online Wissenschaft 2000).

So far no method has been described to reduce or even prevent the release of GMO pollen from crop stands. The present invention now provides a new seed composition as well as a method which enables farmers and researchers to work with GMO crops without the undesired spread of GMO pollen over crops growing on adjacent fields by above described ways and reasons.

The present invention overcomes the above described problems by sowing GMO seeds in a mixture with male fertile or wildtype seeds, so that the total crop stand of male fertile plants will have a normal grain set and yield with reduced release or without the release of GMO pollen.

Male Sterility

Plants can be rendered male sterile by mechanical, chemical or genetic processes. Mechanically and chemically induction of male sterility is very time consuming or often does not fulfil up to now the criteria of low toxicity or reliability respectively. Nuclear male sterility can be obtained by plant transformation techniques but crossing a nuclear male sterile mother leads to a segregation of male sterile and fertile offspring.

Cytoplasmic male sterility (cms) is based on an interaction between mitochondrial genes and nuclear genes, which leads to dysfunctional pollen. Certain nuclear genes can overcome this effect when they are introduced by crossing into cms-plants, restoring the male fertility. Such genetic systems have been introduced in many crop species for the production of cheap hybrid seeds. When cms-maize plants are pollinated by fertile maize plants they often produce higher yields than their isogenic male fertile counterparts (Stamp et al., 2000).

Cultivation of Mixtures from GMO Male Sterile Plants and Fertile Plants in Crop Stands

In dependence or the target trait male sterile GMO plants from species with a sufficiently high pollen shed can be cultivated in random mixtures or row-wise with wild type (wildtype), male fertile plants. The latter can be isogenic or non-isogenic to the above-mentioned GMO plants without reducing the yield potential when pollen release and demand of the mixture is synchronised. It is essential that a male sterile system is chosen, which reliably prevents the development of functional pollen under field conditions.

It is therefore the object of the present invention to reduce or prevent the release of pollen or functional pollen from GMO crop stands by

a) choosing a male sterile version of a GMO crop plant;

b) choosing isogenic or non-isogenic pollen donor plants which can be wildtype and male fertile for the purpose of preventing the release of pollen or functional pollen from GMO plants;

c) growing the GMO male sterile plants and the wildtype pollen donor plants together, in random mixtures or row-wise, thereby allowing for pollination of the GMO male sterile plants by the plants of the wildtype pollen donor.

The novel method of the present invention reduces or prevents the release of GMO pollen from crop stands or prevents or reduces the release of functional GMO pollen from crop stands. Thus, undesirable impacts like the outcrossing to fields with wildtype plants of the same species, a direct unwanted effect of the GMO pollen on other organisms and the content of GMO pollen in natural products like honey are minimized or even avoided. This is achieved by a mixture between a male sterile version of a GMO plant, incapable or less capable of producing pollen or functional pollen, and male fertile, wildtype plants, which will pollinate all plants in this mixture. Such mixtures can be cultivated for crop species, which produce a sufficient surplus of pollen for a high proportion of plants with the GMO trait within said mixture. Such a crop stand will have a normal grain set and yield with reduced or without the release of GMO pollen.

### Definitions

The following terms are defined:

Farmer's field:

This is a community of plants, which are cultivated on a field for agricultural products

Cross-pollination:

the pollination of the ovules of a plant by another plant which is non-isogenic to it, see non-isogenic

GMO (=genetically modified organism):

a state where an organism or a part of it has been genetically modified by introducing DNA fragments by using biotechnological methods

Grains:

seed and caryopses produced as agriculture commodities

Male sterile:

a plant without production of pollen or without production of functional pollen because of mechanical castration or the chemical and genetic induction of sterility in general

Non-isogenic:

a state of genetic dissimilarity between individuals when their nuclear genomes possess less than 87% statistical similarity

Seeds:

seeds and caryopses For reproduction purposes

Wildtype:

A state where an organism has not been genetically modified.

### References

The following references are cited:

Emberlin, J., B. Adams-Groom and J. Tidmarsh 1999: The dispersal of maize (Zea mays) pollen. A report based on evidence available from publications and internet sites. A report comissioned by the soil association: national Pollen Research Unit, University College Worcester, Worcester, UK.

Losey J. E., L. S. Raynor, and P. C. Lyons 1999: Transgenic pollen harms monarch larvae. Nature 399, 214.

Hodges D. 1984: The pollen loads of the honey bee. International Eee Research Association, London.

RP-online Wissenschaft 2000: Gentransfer von Pflanze auf Bakterium. 24.95.00.

Stamp P., S. Chowchong, M. Menzi, U. Weingartner, and O. Kaeser 2000: Increase in the yield of cytoplasmic male sterile maize revisited. Crop Sci. 40, 1586-1587.

Crop species grown for the purpose of grain production such as cereals, rape seed (Brassica napus L.) and sunflower (Halianthus annuus L.) are the major staple food for mankind, important sources of energy-rich fodder, and the basis for plant derived oils. For many crops of international importance such as maize and rape seed, GMO traits have been introduced into commercial plant versions, which, for example, can induce tolerance to pests like the European corn borer.

Crops like maize produce a high surplus of pollen, which can be air-borne in a viable state to considerable distances from the plant direction (Table 1). This example demonstrates that a high number of functional pollen occurs even at long distances from a single plant. This can create major problems when GMO pollen per se or the pollination of wildtype plants is undesirable for reasons described above.

### EXAMPLE 1

It has been demonstrated in field tests that male sterile versions of maize varieties, pollinated by fertile plants of the same plant version, often yield even higher than their fertile versions (Table 2, derived from Stamp et al. 2000). For crops with a sufficient pollen production, for example maize and rape seeds, in isogenic and non-isogenic mixtures with less than 20% fertile plants a reliable pollination of all plants is achieved. For this reason an undesirable release of GMO pollen can be averted when GMO crops are cultivated in a male sterile version, pollinated in a mixture by wildtype male fertile versions. The composition of the seeds for preventing the release of pollen or functional pollen from GMO crops comprises a mixture of 5% to 50% seeds for male fertile plants which are isogenic or non-isogenic to the GMO plant version and 50% to 95% seeds for male sterile female GMO plants. Preferably, the mixture comprises 20% for male fertile plants and 80% for male sterile GMO plants.

### EXAMPLE 2

The composition for reducing the release of pollen from GMO plants may comprise a mixture of 5% to 50% seeds of male fertile GMO pollinator plants together with 50% to 95% seeds for male sterile GMO plants. Preferably these mixtures comprise 20% for male fertile plants and 80% for male sterile plants. In a further embodiment of the present invention the composition may also comprise a mixture of 5% to 25% of seeds of male fertile wildtype pollinator plants together with 5% to 25% of seeds of male fertile GMO plants together with 50% to 90% seeds of male sterile female GMO plants.

This invention can be applied for all crops, which produce a sufficient surplus of pollen for the air-borne or insect-borne pollination of neighbouring plants. It is applicable for GMO target traits, which do not exclude the presence of a wildtype plant like in the case of herbicide tolerance. In such cases the amount of GMO pollen, which is released from farmer's fields can be largely reduced by cultivating mixtures of male sterile GMO plants with male fertile GMO plants, containing less than 50% of the male fertile version.

TABLE 1
Estimated relative concentration and absolute number of
pollen in relation to the distance from the pollen donor
(Emberlin et al. 1999)

| | Pollen concentration | Absolute pollen number |
|---|---|---|
| | in the air (100 = | from a single plant |
| Distance from the | concentration in 1 m | (basis 25 million |
| pollen donor | distance from the | pollen grains released |
| (downwind) | plant) | from one plant) |
| 60 m | 2% | 500 000 |
| 200 m | 1.1% | 275 000 |
| 500 m | 0.5-0.75% | 125 000-187 500 |

TABLE 2
Grain biomass yield (g per m²) of the male sterile cms
Swiss hybrids Corso and Silex at two plant densities (plants per
m²) averaged over two years. Changes (%) in relation to the
fertile version are presented in parenthesis.

| | Density | Yield |
|---|---|---|
| Corso | 9 | 961 (9.7) |
| Corso | 12 | 1029 (19.3) |
| Silex | 9 | 934 (3.9) |
| Silex | 12 | 942 (3.1) |

1. A seed composition for reducing or preventing the release of pollen from genetically modified (GMO) plants or for reducing or preventing the release of functional GMO pollen from said plants, comprising a mixture of 5% to 50% seeds for male fertile pollinator plants and 50% to 95% seeds for male sterile female GMO plants with target GMO traits, isogenic or non-isogenic to said male fertile pollinator plants.

2. The seed composition according to claim 1, comprising a mixture of 20% seeds for male fertile pollinator plants and 80% seeds for male sterile GMO plants.

3. A method for culturing GMO plants, characterized in that said method reduces or prevents the release of GMO pollen or reduces or prevents the release of functional GMO pollen without loss of grain yield, said method comprising the steps of:
i. selecting a seed mixture of a male sterile plant version bearing target GMO traits and one or more male fertile isogenic or non-isogenic plant versions; and
ii. sowing said seeds of both isogenic or non-isogenic plant versions thereby allowing for pollination of GMO male sterile plants.

4. The method according to claim 3, comprising the use of a mixture of 5% to 50% seeds for male fertile pollinator plants and 5% to 95% seeds for male sterile GMO plants.

5. The method according to claim 4, comprising the use of a mixture of 20% seeds for male fertile pollinator plants and 80% seeds for male sterile female GMO plants.

6. The method according to claims 3 to 5 for preventing the release of GMO pollen or functional GMO pollen, wherein said male fertile pollinators are wildtype plants.

7. The method according to, claims 3 to 5 for reducing the release of GMO pollen or functional GMO pollen, wherein said male fertile plants are GMO plants.

8. The method according to claims 3 to 5 for reducing the release of GMO pollen or functional GMO pollen, wherein said male fertile plants comprise a mixture of male fertile GMO plants and male fertile wildtype plants.

9. The method according to claim 8 comprising the sowing of a mixture of 5% to 25% of seeds of male fertile wildtype pollinator plants and 5% to 25% of seeds of male fertile GMO pollinator plants together with 50% to 90% seeds of male sterile female GMO plants.

10. The method according to claims 8 and 9, comprising the use of 10% seeds for male fertile wildtype pollinator plants, 10% seeds for male fertile GMO plants and 80% seeds for male sterile female GMO plants.

11. The method according to claims 3 to 10, wherein a mixture of isogenic or non-isogenic plant versions is randomly sown.

12. The method according to claims 3 to 10, wherein the isogenic or non-isogenic plant versions are sown in separate rows.

13. A method for planting a field according to one of the claims 3 to 12 comprising the steps of planting within a field:
i) male sterile seeds having target GMO traits to produce male sterile female plants that do not release pollen or functional pollen;
ii) the seeds of one or more male fertile plant versions, isogenic or non-isogenic to the GMO plants, for pollination of said male sterile GMO plants;
iii) permitting a high grain biomass yield of both plant versions without release or with reduced release of GMO pollen or functional GMO pollen.
